(19) United States
(12) Patent Application Publication
(10) Pub. No.: US 2012/0116485 A1
(43) Pub. Date: May 10, 2012

(54) PHOTOTHERAPY DEVICE FOR ILLUMINATING THE PERIPHERY OF A WOUND AND PHOTOTHERAPY SYSTEM INCORPORATING THE SAME

(75) Inventor: Tom Burgmann, Mississauga (CA)

(73) Assignee: MEDX HEALTH CORP., Mississauga (CA)

(21) Appl. No.: 13/151,521

(22) Filed: Jun. 2, 2011

Related U.S. Application Data
(63) Continuation of application No. 12/918,902, now abandoned.
(60) Provisional application No. 61/064,198, filed on Feb. 21, 2008.

(51) Int. Cl. A61N 5/06

(52) U.S. Cl. 607/90

(57) ABSTRACT

A phototherapy device comprises a plurality of radiation emitting sources arranged at spaced locations along at least a portion of the periphery of a wound to be treated and a controller communicating with and controlling operation of the radiation emitting sources.

### FIELD OF THE INVENTION

The present invention relates generally to therapeutic devices and in particular, to a phototherapy device for illuminating the periphery of a wound and to a phototherapy system incorporating one or more such phototherapy devices. The present invention also relates to a wound sensing device and to a method of treating a wound.

### BACKGROUND OF THE INVENTION

Wounds have commonly been treated by covering them with bandages, gauze or other suitable flexible, sterile materials which tend to block exposure of the wounds to natural light. Unfortunately, contrary to this common practice, medical research and literature have shown a positive correlation to the healing process in animal and human tissue repair when exposed to narrow band light.

Many phototherapy techniques for applying light to an area of a subject to be treated have been considered. For example, U.S. Pat. No. 5,616,140 to Prescott discloses a battery operated, portable laser bandage having one or many lasers or hyper-red light emitting diodes imbedded therein to be worn by a patient and applied to a specific treatment area. The bandage supplies the patient with a preprogrammed laser therapy regimen. The patient may wear the bandage for up to a week between visits to a physician. At the end of the prescribed treatment length or at the end of the week, batteries in the bandage may be changed or recharged and the physician may re-program the bandage for a different laser therapy regimen, if desired.

U.S. Pat. No. 6,443,978 to Zharov discloses a device for the physiotherapeutic irradiation of spatially extensive pathologies by light. The device comprises a matrix of optical radiation sources such as lasers or light emitting diodes placed on the surface of a substrate having a shape that generally conforms to the shape of the pathology to be treated. In addition, the device contains stops and a holder to fix the substrate against the bioobject. Additional modules are provided to adjust the temperature, pressure and gas composition over the pathology to be treated.

U.S. Pat. No. 7,081,128 to Hart et al. discloses a device to be placed in direct skin contact and surround an injured area to be treated. The device comprises a therapeutic light source including a multiplicity of light emitting diodes (LEDs) having wavelengths in the ranges of 350 nm to 1000+nm. A neoprene-type or other non-allergenic material is used to set arrays of LEDs in layers at different spacings from the skin tissue. The distances of the various arrays of LEDs from the skin tissue vary from contact or near contact to several millimeters. Each LED array is independently controlled allowing for optimal modulation of light frequencies and wavelengths. Technology is integrated allowing for biomedical feedback of skin tissue temperature and other statistical information. A low voltage, portable power supply and an analog/digital, input/output connection device are integrated into the device.

U.S. Patent Application Publication No. 2004/0166146 to Holloway et al. discloses a phototherapy bandage capable of providing radiation to a localized area of a patient for accelerating wound healing and pain relief, providing photodynamic therapy, and for aesthetic applications. The phototherapy bandage may include a flexible light source that is continuous across the bandage and that outputs selected light, such as visible light, near-infrared light or other light. The intensity of the output light is substantially constant across the bandage. The phototherapy bandage may also be flexible and capable of being attached to a patient without interfering with the patient's daily routine. The phototherapy bandage may conform to the curves of the patient and may come in a variety of shapes and sizes.

U.S. Patent Application Publication No. 2006/0173253 to Ganapathy et al. discloses a fluid blood detection system that is operable in conjunction with a reduced pressure wound treatment (RPWT) system, as well as with ancillary therapy and monitoring systems applied concurrently with the RPWT system. The fluid blood detection system operates by optically characterizing the content of wound fluids to the extent of identifying percentage blood content. This identification relies upon the transmission of select wavelengths of light across a volume of wound fluid to a photodetector connected to signal processing instrumentation capable of quantifying the absorption characteristics of the wound fluid. The photodetector may be implemented in conjunction with either a fluid flow conduit (i.e. reduced pressure tubing directing wound fluid away from the wound dressing) or more directly in association with the materials that comprise the wound dressing positioned within the wound bed itself. In addition, the fluid blood detection system is configured to operate in conjunction with blood gas monitoring systems operating with the RPWT system.

U.S. Patent Application Publication No. 2006/0173514 to Biel et al. discloses a light emitting treatment device including one or more light members, which are configured to emit light energy for the purpose of performing localized photodynamic therapy at a targeted field. The light members may be disposed in a substantially uniform array and be configured to emit light energy in a substantially uniform pattern. The light emitting treatment device has a self-contained energy supply and may be controlled to deliver one or more various light doses and dose rates at various light frequencies per treatment. The light emitting treatment device may be made of a polymeric material configured to conform to a body surface. The light emitting treatment device may further include a heat dissipating layer such as a layer of gold or gold alloy, or a layer of adhesive.

U.S. Patent Application Publication No. 2006/0217787 to Olson et al. discloses a light therapy device comprising a light source for delivering light energy to a portion of a patient's body. The light source comprises one or more light emitters for providing input light. A light coupling means directs the input light into a light guide comprising flexible optically transparent light guide material. A light extraction means is applied to a surface of the light guide material. The light extraction means is positioned to provide light therapy treatment to one or more localized areas of the patient's body. A control means controls light dosage relative to intensity, wavelength, modulation frequency, repetition, and timing of treatments.

As will be appreciated, the above-described phototherapy devices show a variety of techniques to deliver light to the area of the subject to be treated. Unfortunately however, these phototherapy devices have been found to be less than ideal in terms of ability to sense the wound healing process. Although wound sensing techniques do exist, prior art wound sensing has revealed some common trends. Much of the work carried out in wound sensing has focused on biochemical assays and wound progression metrics, such as wound size and coloration rather than monitoring factors that contribute directly to wound formation such as wound-site pressure. As is known, common pressure wounds and wounds due to peripheral vascular disorder form due to pressure and bony protrudances in the body. Monitoring patient activity at high risk sites on the body is a difficult task requiring regular observation by clinical staff.

Although patient monitoring systems and devices have been considered, these systems and devices have proven to be unsatisfactory as they do not take into account the pressure of wound tissue or mobile long-term monitoring for patients. For example, U.S. Pat. No. 6,840,117 to Hubbard Jr. discloses a patient monitoring system including a replaceable laminar sensor to be placed on a bed, the sensor including distributed force sensing elements providing output signals to processing apparatus including a near-bed processor and a central processor coupled to the near-bed processor by a wireless communication link. The processing apparatus applies spatial weighting to the sensor output signals to derive the force distribution across the sensor, and processes the force distribution over time to generate patient status information such as patient presence, position, agitation, seizure activity, respiration, and security. This information can be displayed at a central monitoring station, provided to a paging system to alert attending medical personnel, and used to update medical databases. The sensor may be manufactured from layers of olefin film and conductive ink to form capacitive sensing elements.

U.S. Pat. No. 7,276,917 to Deangelis et al. discloses a a flexible, resilient capacitive sensor suitable for large-scale manufacturing. The sensor includes a dielectric, an electrically conductive detector and trace layer on the first side of the dielectric layer including a detector and trace, an electrically conductive reference layer on a second side of the dielectric layer, and a capacitance meter electrically connected to the trace and to the conductive reference layer to detect changes in capacitance. The sensor is shielded to reduce the effects of outside interference.

U.S. Patent Application Publication No. 2006/0052678 to Drinan et al. discloses systems and techniques for monitoring hydration. In one implementation, a method includes measuring an electrical impedance of a region of a subject to generate an impedance measurement result, and wirelessly transmitting the data to a remote apparatus. The probe with which impedance is measured may in the form of a patch adhesively secured to the subject.

Notwithstanding the above techniques for phototherapy and patient monitoring, improvements in phototherapy devices and wound sensing devices are desired. It is therefore an object of the present invention to provide a novel phototherapy device for illuminating the periphery of a wound and a phototherapy system incorporating one or more such phototherapy devices. It is also an object of the present invention to provide a novel wound sensing device and method of treating a wound.

### SUMMARY OF THE INVENTION

Accordingly, in one aspect there is provided a phototherapy device comprising:

a plurality of radiation emitting sources arranged at spaced locations along at least a portion of the periphery of a wound to be treated; and

a controller communicating with and controlling operation of said radiation emitting sources.

According to another aspect there is provided a phototherapy system comprising:

at least one computing station; and

one or more phototherapy devices as described above communicating with said at least one computing station.

According to yet another aspect there is provided a method of treating a wound comprising irradiating the skin tissue adjacent the periphery of the wound with light energy at intervals.

According to still yet another aspect there is provided a wound sensing device comprising:

a plurality of sensors for monitoring at least one wound parameter to be positioned adjacent a wound; and

a controller communicating with and reading said sensors.

According to still yet another aspect there is provided a phototherapy bandage comprising:

an upper layer;

a lower layer; and

a plurality of spaced light emitting devices arranged in a ring and positioned between said upper and lower layers.

According to still yet another aspect there is provided a phototherapy bandage comprising:

an upper layer;

a lower layer; and

a plurality of spaced sensors arranged in a ring and positioned between said upper and lower layers.

### BRIEF DESCRIPTION OF THE DRAWINGS

Embodiments will now be described more fully with reference to the accompanying drawings in which:

FIG. 1 shows a phototherapy device comprising a phototherapy bandage and a controller connected to the phototherapy bandage;

FIG. 2 is a top plan view of an emitter and sensor assembly forming part of the phototherapy bandage of FIG. 1;

FIG. 3 is a side view of the emitter and sensor assembly of FIG. 2;

FIG. 4 is an enlarged side view of a portion of the emitter and sensor assembly of FIG. 2;

FIG. 5 is a schematic block diagram of the emitter and sensor assembly of FIG. 2;

FIG. 6 is a cross-sectional view of the phototherapy bandage of FIG. 1 being applied to a wound to be treated;

FIG. 7 is a schematic block diagram of the controller of FIG. 1;

FIG. 8 is a schematic diagram of a phototherapy system employing one or more phototherapy devices;

FIG. 9 is a data record displayed by the phototherapy system of FIG. 9;

FIG. 10 is a top plan view of an alternative emitter and sensor assembly;

FIG. 11 is a perspective view taken from above and from the side of an alternative phototherapy bandage;

FIG. 12 is a perspective view taken from below and from the side of the phototherapy bandage of FIG. 11 being applied to a wound to be treated;

FIG. 13 is a cross-sectional view of the phototherapy bandage of FIG. 12;

FIG. 14 is a perspective view taken from below and from the side of yet another phototherapy bandage;

FIG. 15a is a cross-sectional view of a pressure sensor; and

FIG. 15b is a cross-sectional view of an alternative pressure sensor.

### DETAILED DESCRIPTION OF THE EMBODIMENTS

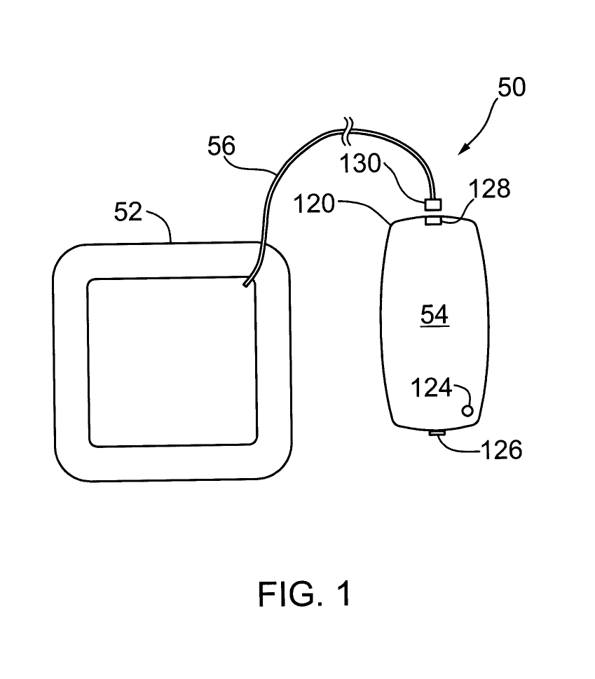

Turning now to FIG. 1, a phototherapy device is shown and is generally identified by reference numeral 50. As can be seen, phototherapy device 50 comprises a phototherapy bandage 52 to be applied to a patient and cover a wound or other pathology to be treated and a controller 54 releasably connected to the phototherapy bandage 52 by a multi-conductor cable 56. In this embodiment, the phototherapy bandage 52 is designed to illuminate the periphery of the wound covered by the phototherapy bandage thereby to promote the healing process without disturbing the dressing overlying the wound bed. The controller 54 provides the operating power for the phototherapy bandage 52 and controls the operation of the phototherapy bandage so that the phototherapy bandage 52 subjects the wound to the desired phototherapeutic treatment regime. The phototherapy bandage 52 and the controller 54 are portable and lightweight allowing the phototherapy device 50 to be worn by a patient without affecting the patient's daily routine. Further specifics of the phototherapy device 50 will now be described.

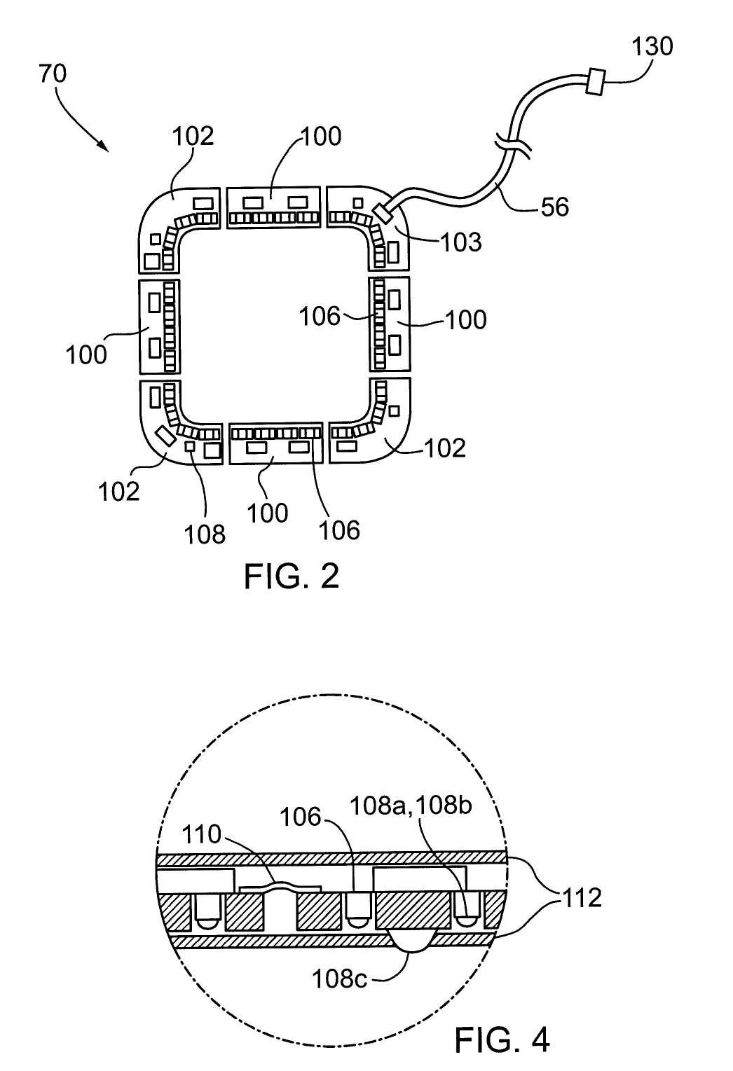

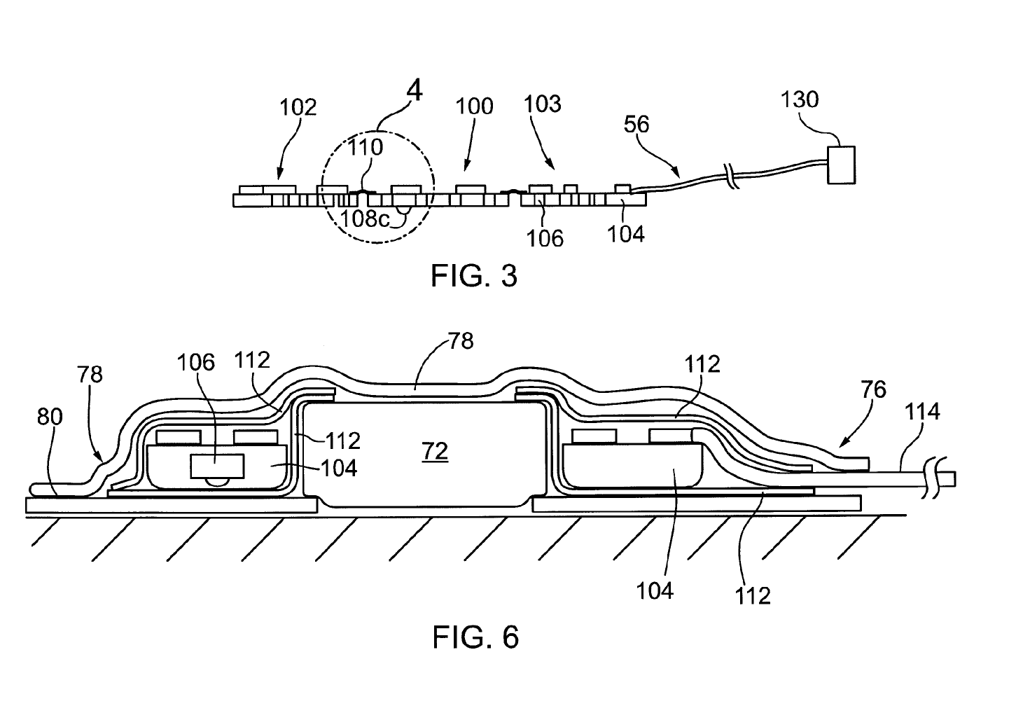

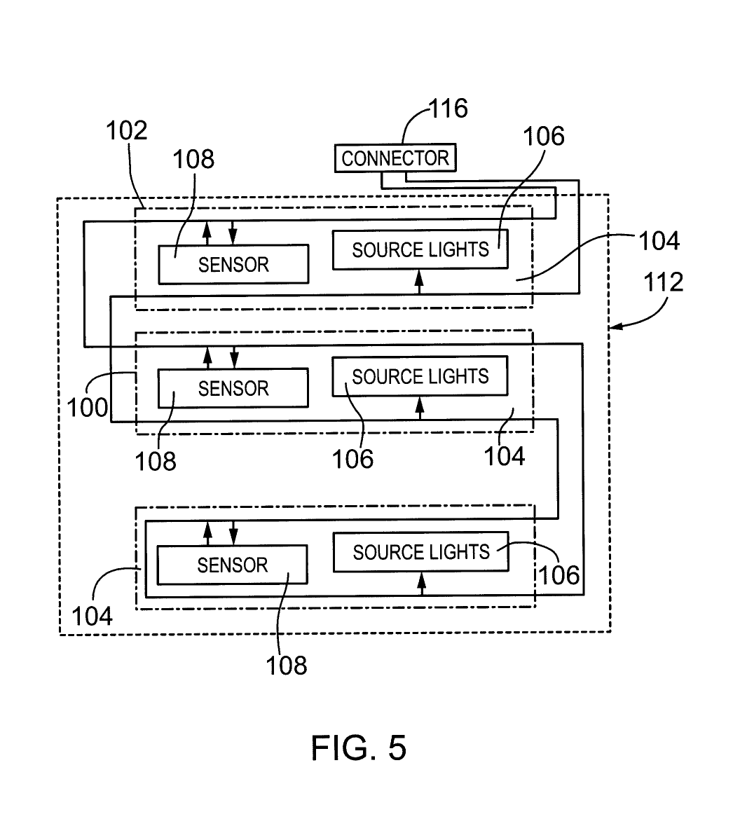

FIGS. 2 to 6 better illustrate the phototherapy bandage 52. As can be seen, the phototherapy bandage 52 comprises an emitter and sensor assembly 70 in the shape of a ring that surrounds a simple or complex dressing 72 sized to overlay the wound bed. The dimension and shape of the ring is selected so that the emitter and sensor assembly 70 surrounds the periphery of the wound and is spaced from the edges of the wound by a distance in the range of from about 1 cm to about 3 cm. The emitter and sensor assembly 70 and the dressing 72 are accommodated in a breathable pouch 76 thereby to promote airflow through the phototherapy bandage 52. Pouch 76 comprises a perforated upper layer 78 and a lower adhesive layer 80 to affix the pouch 76 to the patient. The adhesive layer 80 has a cut-out therein sized to expose the dressing 72 so that the dressing can be brought into direct contact with the wound bed when the phototherapy bandage 52 is applied to the patient. The upper and lower layers 78 and 80 are formed of biologically safe material to inhibit the pouch 76 from adversely affecting the wound or surrounding tissue.

The emitter and sensor assembly 70 comprises a plurality of segments electrically connected in series, with each segment having one of two (2) shapes. In this embodiment, the emitter and sensor assembly 70 comprises four (4) straight segments 100, three (3) curved segments 102 and one (1) curved segment 103. Curved segment 103 differs from the curved segments 102 in that one end of the cable 56 is permanently affixed thereto thereby to connect electrically the emitter and sensor assembly 70 to the controller 54.

The straight and curved segments 100, 102 and 103 are arranged in an alternating pattern thereby to form a generally rectangular ring. Aside from shape, the segments are virtually identical. In this embodiment, each segment 100, 102 and 103 comprises a short, rigid printed circuit board 104. A row of spaced radiation emitting sources 106, in this case four (4) radiation emitting sources, is surface mounted on each printed circuit board 104 at locations so that when the phototherapy bandage 52 is applied to the patient, the radiation emitting sources 106 are aimed at and positioned proximate to the patient's skin tissue. The radiation emitting sources 106 in this embodiment are red, solid-state, light emitting diodes (LEDs) that emit visible light having a wavelength in the range of from about 630 nm to about 690 nm as wound healing is expected to occur primarily in the epidermis and shallow musculoskeletal regions.

Each segment also comprises a plurality of sensors. In particular, in this embodiment, a temperature sensor 108a, a photoreceptor 108b having appropriate spectral filtering and a contact sensor 108c are also surface mounted on the printed circuit board 104. The temperature sensors 108a measure the temperature of the skin tissue at a location proximate the periphery of the wound. Temperature changes provide an indication as to whether the wound is receiving sufficient blood flow and microcirculation or if blood flow is affected by an infection. The photoreceptors 108b measure light emitted by the LEDs 106 that has entered the skin tissue surrounding the wound and has backscattered into the wound bed as a result of cellular membranes. The amount of backscattered light received by the photoreceptors 108b provides information concerning the healing stage of the wound. Pairs of contact sensors 108c are used to measure electrical impedance across the wound. Measuring electrical impedance provides an indication of the moisture content in the vicinity of the wound bed allowing situations where the wound fluid has saturated the dressing 72 and leaked outside the periphery of the wound bed to be detected so that appropriate steps can be taken to change the dressing 72.

Flexible, insulated multi-conductor cables 110 interconnect adjacent segments electrically and mechanically. Use of the flexible cables 110 permits the segments 100, 102 and 103 to take on various angles and to move relative to one another. In this manner, when the phototherapy bandage 52 is applied to a patient, each segment can take on an orientation independent of the other segments. This allows the LEDs 106 to remain generally coplanar with the tissue surrounding the wound even when the underlying tissue is flexed by muscular, tendon or fat movement. A biologically safe, translucent material 112 encapsulates the segments 100, 102 and 103 and the cables 110 to provide the emitter and sensor assembly 70 with a smooth patient contact surface that does not adversely affect the wound or surrounding tissue.

The controller 54 comprises an outer housing 120 that is accommodated by a disposable outer sleeve 122 formed of biologically safe material. The outer sleeve 122 has an adhesive coating covered by a release layer (not shown) that can be removed to expose the adhesive coating thereby to allow the controller 54 to be affixed to the patient adjacent the phototherapy bandage 52. A light emitting diode (LED) 124 and a switch 126 are provided on the housing 120. The LED 124 provides a user with visual operational feedback. A connector 128 on the housing 120 receives a low profile connector 130 at the opposite end of the cable 56. The interior of the housing 120 accommodates a printed circuit board 132 on which the controller electronics are mounted.

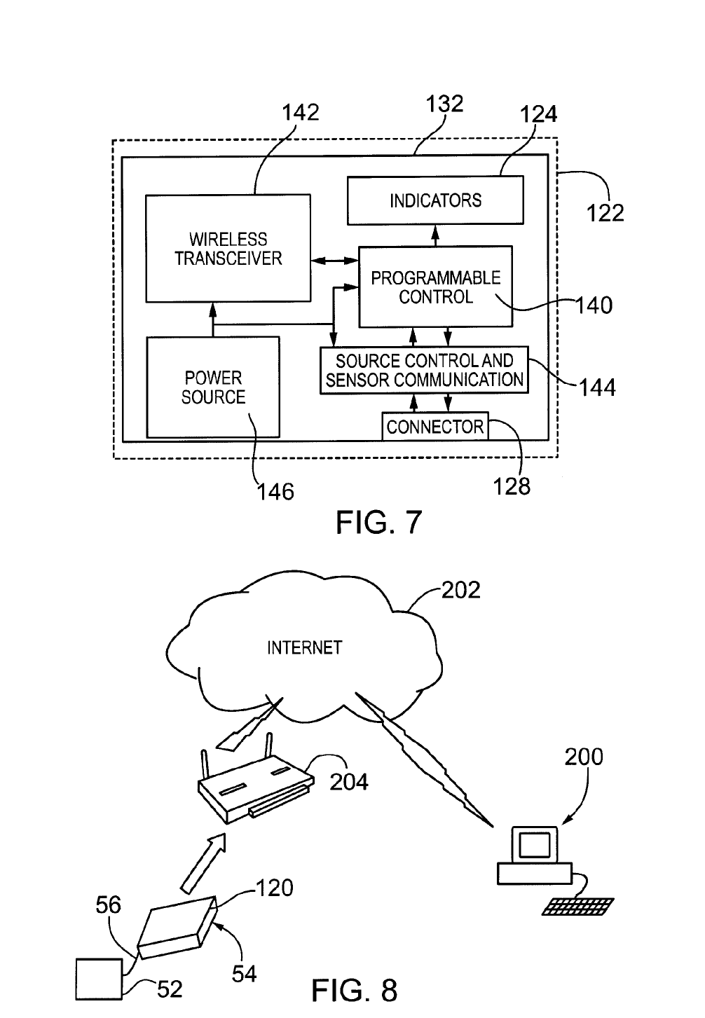

FIG. 7 best illustrates the controller electronics. As can be seen, the controller electronics comprise a microprocessor 140, a wireless communications transceiver 142 to enable bi-directional communications with remote devices, a driver 144 that is responsive to the microprocessor 140 to control operation of the LEDs 106, temperature sensors 108a, photoreceptors 108b and contact sensors 108c, and random access memory (RAM) (not shown). A power source 146 provides operating power to the microprocessor 140, wireless communications transceiver 142 and driver 144. The power source 146 comprises one or more chargeable or rechargeable batteries. The number and type of batteries are selected to enable the controller 54 to operate the phototherapy bandage 52 for extended periods of time thereby to ensure that the phototherapy bandage 52 functions over the intended phototherapeutic treatment regime. If desired, the power source 146 may comprise other components to supplement the batteries such as for example, ultra capacitors. In this manner, very high instantaneous output currents may be realized allowing the controller 54 to operate the LEDs 106 at higher peak output levels as well as to drive larger rings of segments. Alternatively, the power source 146 may comprise a transformer and regulator to convert power from a conventional ac mains supply to the appropriate operating power for the microprocessor 140, wireless communications transceiver 142 and driver 144.

The RAM stores one or more phototherapy treatment protocol programs that can be executed by the microprocessor 140 to control the operation of the phototherapy bandage 52. The phototherapy treatment protocol program that is being executed by the microprocessor 140 determines the nature, timing and duration of the phototherapeutic treatment regime to which the wound is subjected. In particular, the phototherapy treatment protocol program that is being executed determines the intervals at which power is supplied to the segments by the driver 144 to illuminate the LEDs 106, the duration the LEDs 106 are powered, the pattern by which the LEDs 106 are powered and the intensity level at which the LEDs 106 are operated. The phototherapy treatment protocol program also determines the intervals at which the outputs of the temperature sensors 108a, photoreceptors 108b and contact sensors 108c are read by the microprocessor 140 and stored in the RAM.

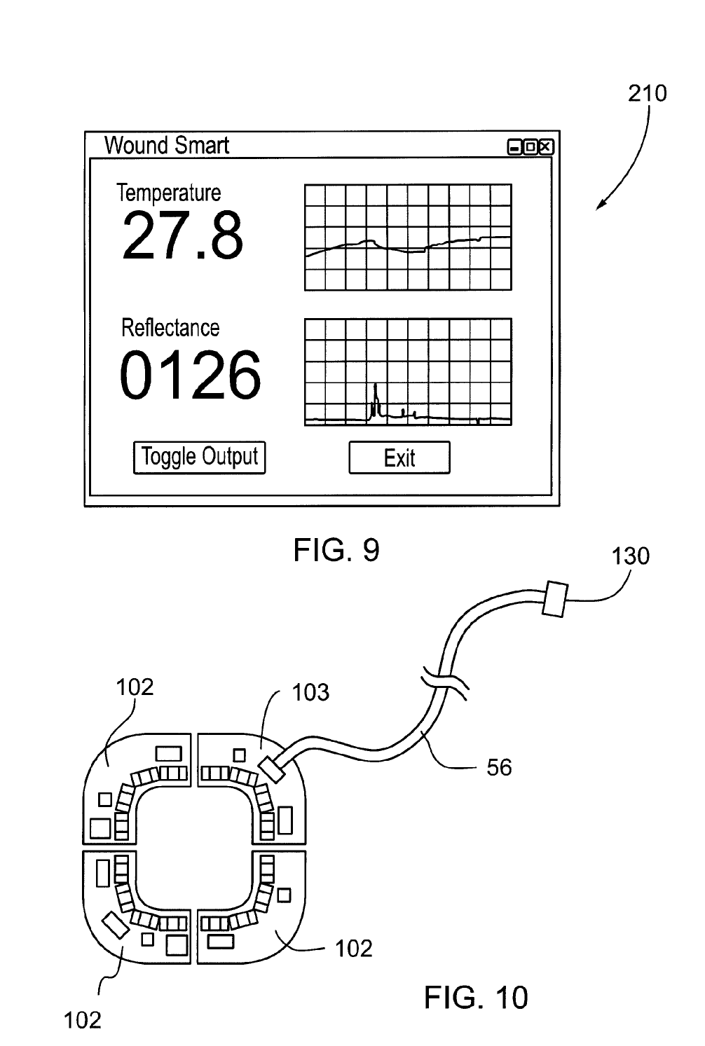

The wireless communications transceiver 142 allows the controller 54 to communicate with remote devices such as for example personal digital assistants (PDAs), cellular telephones, laptop computers, tablet PCs or other computers and other processing devices via a wireless communications link (radio frequency (RF), infrared etc.) using a suitable wireless protocol such as for example, Zigbee, Bluetooth, WiFi, MICS, ANT etc. In this manner, the phototherapy treatment protocol programs stored in the RAM can be updated allowing the phototherapy bandage 52 to operate according to different phototherapeutic treatment regimes. The read temperature, light and impedance data stored in the RAM can also be communicated to a remote computing device allowing the temperature, light and impedance data to be analyzed and displayed. For example, FIG. 8 shows the phototherapy device 50 communicating with a remote computing station 200 over an Internet connection 202 via a wireless modem 204. The remote computing station 200 executes a program to analyze the temperature, light and impedance data received from the controller 54 and present the results of the analysis graphically. FIG. 9 is a data record 210 displayed by remote computing station 200. In this example, the data record 210 comprises a graph of the temperature readings recorded by the phototherapy device 50 and the average recorded temperature. The data record also comprises a graph of reflectance readings recorded by the phototherapy device 50 and the average recorded reflectance. Of course, other data records presenting different data can be displayed.

As will be appreciated by those of skill in the art, although only one phototherapy device 50 is shown communicating the remote computing station 200, in typical situations, the remote computing station 200 collects data from a significant number of phototherapy devices 50. In this manner, over time, recorded data from different phototherapy devices and patients can be used to establish acceptable wound healing profiles. With acceptable wound healing profiles known, a wound covered by a phototherapy bandage 52 can be assessed simply by examining the recorded temperature, light and impedance data retrieved from the phototherapy bandage 52. This allows the wound to be assessed remotely without requiring the phototherapy bandage 52 to be removed from the patient reducing the burden on medical personnel. Recorded temperature, light and impedance data that deviate from the acceptable wound healing profiles can be detected and used to generate an alarm or other indicator.

The phototherapy device 50 is intended to be used in a manner following standard wound assessment and treatment methods currently followed by medical personnel. When a patient suffers a wound, assuming the wound has been cleansed, debrided and/or otherwise treated, a phototherapy bandage 52 having segments that form a ring large enough to surround the wound is selected. The selected phototherapy bandage 52 is then applied to the patient so that the dressing 72 overlies the wound bed allowing the dressing 72 to absorb exudate fluid. The adhesive layer 80 maintains the phototherapy bandage 52 in position. Of course, additional adhesive tape may be used to supplement attachment of the phototherapy bandage 52 to the patient. Once the phototherapy bandage 52 has been properly affixed to the patient, the connector 130 on the cable 56 is brought into engagement with the connector 128 on the controller housing 120. The controller 54 is then turned on by operating the switch 126 and the controller is placed in the disposable sleeve 122 and affixed to the patient at a location proximate the phototherapy bandage 52.

Once turned on, the microprocessor 140 executes the selected phototherapy treatment protocol program. When the phototherapy treatment protocol program signifies the start of an LED illumination interval, the microprocessor 140 signals the driver 144. The driver 144 in response provides operating power to the emitter and sensor assembly 70 causing the LEDs 106 of the segments 100, 102 and 103 to illuminate at the desired intensity level. As the LEDs 106 are oriented towards the skin tissue, the periphery of the wound is subjected to light having a wavelength designed to promote wound healing. Thus, the periphery of the wound is subjected to timed doses of light selected to affect growth factors, microcirculation and angiogenesis positively as well as to promote the natural healing process. With the wound subjected to emitted light, the temperature sensors 108a measure the temperature adjacent the wound. The photoreceptors 108b measure light backscattered through the wound bed. Pairs of contact sensors 108c at diametric locations along the ring of segments measure the impedance across the wound bed. The output of the temperature sensors 108a, the output of the photoreceptors 108b and the output of the pairs of contact sensors 108c are read by the microprocessor 140 at intervals during execution of the phototherapy treatment protocol program and stored in the RAM. At the end of the interval, the driver 144 isolates the emitter and sensor assembly 70 from the operating power so that the LEDs 106 turn off. During gaps between LED illumination intervals, the controller electronics are conditioned to a sleep mode to conserve power. The above process is performed for each LED illumination interval. The read temperature, light and impedance data stored in the RAM is transmitted to the remote computing station 200 at intervals under the control of the microprocessor 140. Of course, if desired the microprocessor 140 can be programmed so that it only transmits the read temperature, light and impedance data in response to requests received from the remote computing station 200.

Although the controller 54 is described as illuminating all of the LEDs 106 continuously during the LED illumination intervals, if desired, the LEDs 106 can be turned on and off during the LED illumination intervals according to a duty cycle. Also, the LEDs 106 of different segments can be illuminated at different times to reduce peak level power drawn from the power source 146.

The phototherapy bandage 52 in this embodiment is intended for single patient use and is disposed of at the conclusion of phototherapeutic treatment regime. The controller 54 is however reused.

If desired, the emitter and sensor assembly 70 may comprise LEDs 106 that operate at different wavelengths. In this case, the photoreceptors 108b measure the amount of backscattered light at each frequency allowing changes in wound color to be detected. Knowing the color of the wound allows the stage (i.e. blood filled (very red), pre-scab (white) and hard scab (brown)) of wound healing to be identified.

Although the emitter and sensor assembly 70 is described and shown as comprising eight (8) segments shaped and arranged to form a generally rectangular ring, those of skill in the art will appreciate that other segment configurations are possible. The number of segments employed is generally a function of the size of the wound over which the phototherapy bandage 52 is placed. For smaller wounds, the emitter and sensor assembly 70 may comprise fewer segments. For example, as can be seen in FIG. 10, an emitter and sensor assembly 70 comprising only four (4) curved segments 102 and 103 is shown. For larger wounds, the emitter and sensor assembly 70 may comprise more segments. For most wound situations, it is anticipated that phototherapy bandages 52 having emitter and sensor assemblies 70 comprising either four (4), six (6) or eight (8) segments will be suitable as the segment rings of such phototherapy bandages encompass areas equal to approximately 4 cm², 8 cm² or 18 cm² respectively. Of course, depending on the shape of the wound, the number of straight segments and curved segments that are used may be varied. Also, the segments forming the emitter and sensor assembly 70 need not be arranged to form an enclosed ring. For example, the segments can be arranged in a C-shaped configuration, in a linear strand or other suitable configuration. In such cases, as will be appreciated, the segments will extend along only a portion of the wound periphery.

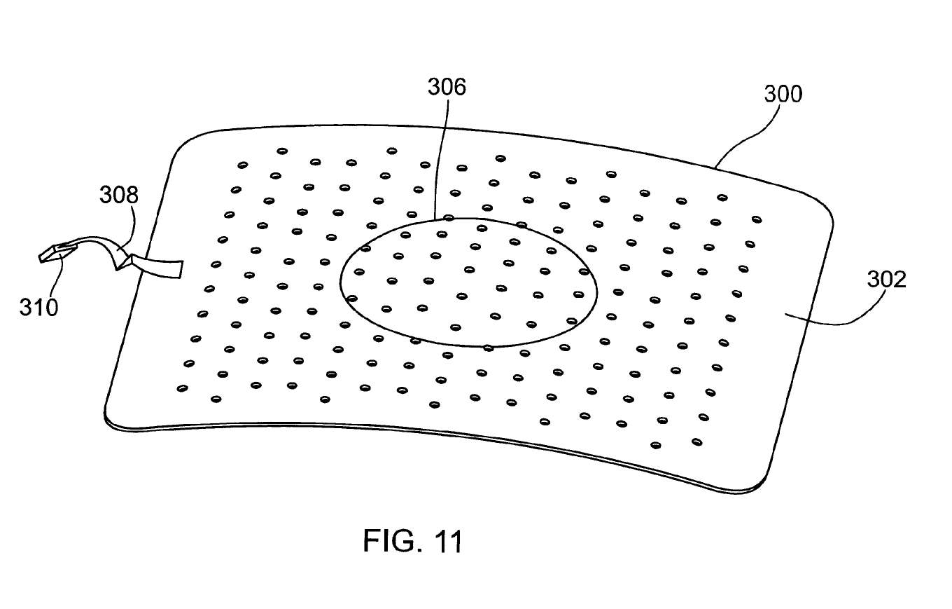

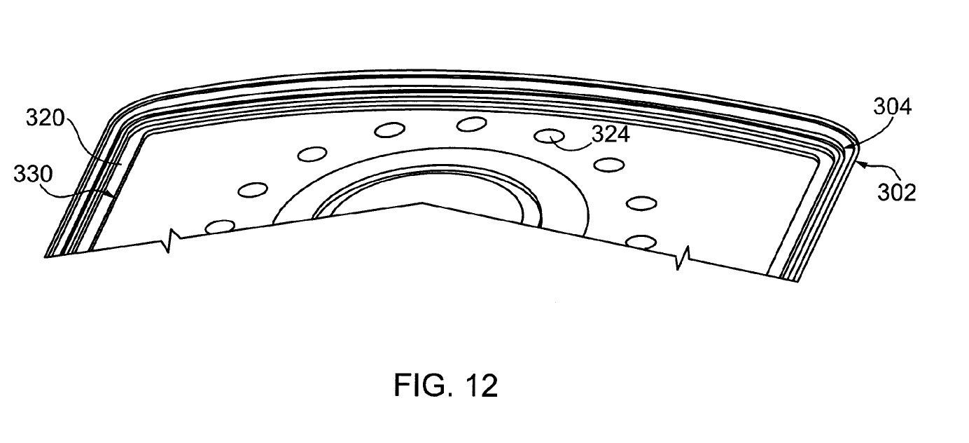

Although the use of segments interconnected by flexible cables allows the LEDs 106 to remain generally coplanar with the skin tissue surrounding the wound even though the LEDs 106 are mounted on rigid printed circuit boards, alternative phototherapy bandage structures can be employed. For example, turning now to FIGS. 11 to 13, another embodiment of a phototherapy bandage is shown and is generally identified by reference number 300. In this embodiment, the phototherapy bandage 300 is of a multilayer construction and comprises an upper perforated breathable layer 302 disposed on one side of an absorbent layer 304 formed of gauze or other suitable material. The breathable layer 302 has a centrally located, circular raised portion 306 formed thereon. A cable 308 having a connector 310 at one end extends through the breathable layer 302. The connector 310 mates with the connector 128 on the controller housing 120.

A flexible printed circuit board 320 is disposed on the other side of the absorbent layer 304 and has a circular cut-out 322 therein that is generally aligned with the raised portion 306. The printed circuit board 320 is of a polymide and copper multilayer construction. Red LEDs 324 are surface mounted on the printed circuit board 320 about the periphery of the cut-out 322. A temperature sensor 326, a photoreceptor 328 and contact sensors 329 are also surface mounted on the printed circuit board 320 adjacent the cut-out 322. The cable 308 is permanently affixed to the printed circuit board at its other end allowing the controller 54 to control the operation of the phototherapy bandage 300. An adhesive layer 330 is provided beneath the printed circuit board 320. The adhesive layer 330 is formed of biologically safe material and is designed to contact the patient directly thereby to affix the phototherapy bandage 300 to the patient. A circular cut-out 332 that is generally aligned with the raised portion 306 is also provided in the adhesive layer 330. As will be appreciated, the cut-outs 322 and 332 are dimensioned so that the wound bed is not contacted by the adhesive layer 330 or the printed circuit board 320. In this manner, when the phototherapy bandage 300 is applied to a patient to cover a wound, the wound bed is only covered by the breathable and absorbent layers 302 and 304. If desired separate dressing material may be provided in the cut-out region to overlie the wound bed and isolate the absorbent layer 304 from direct contact with the wound bed.

The phototherapy bandage 300 is responsive to the controller 54 and operates in a manner similar to the phototherapy bandage 52. During execution of a phototherapy treatment protocol program by the microprocessor 140, at the start of an LED illumination interval, the microprocessor 140 conditions the driver 144 to provide an operating voltage to the LEDs 324 so that the LEDs 324 are illuminated at the desired intensity levels. The microprocessor 140 also reads the outputs of the temperature sensor 326, photoreceptor 326 and contact sensors 329 and stores the read temperature, light and impedance data in the RAM.

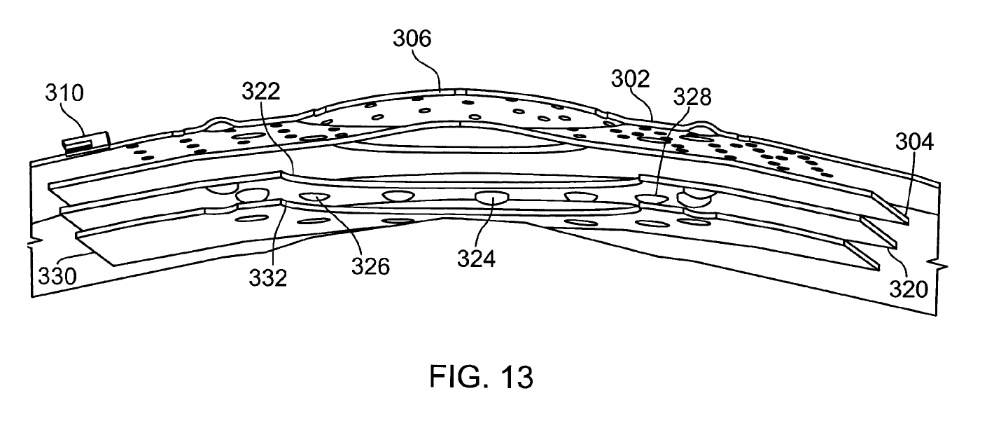

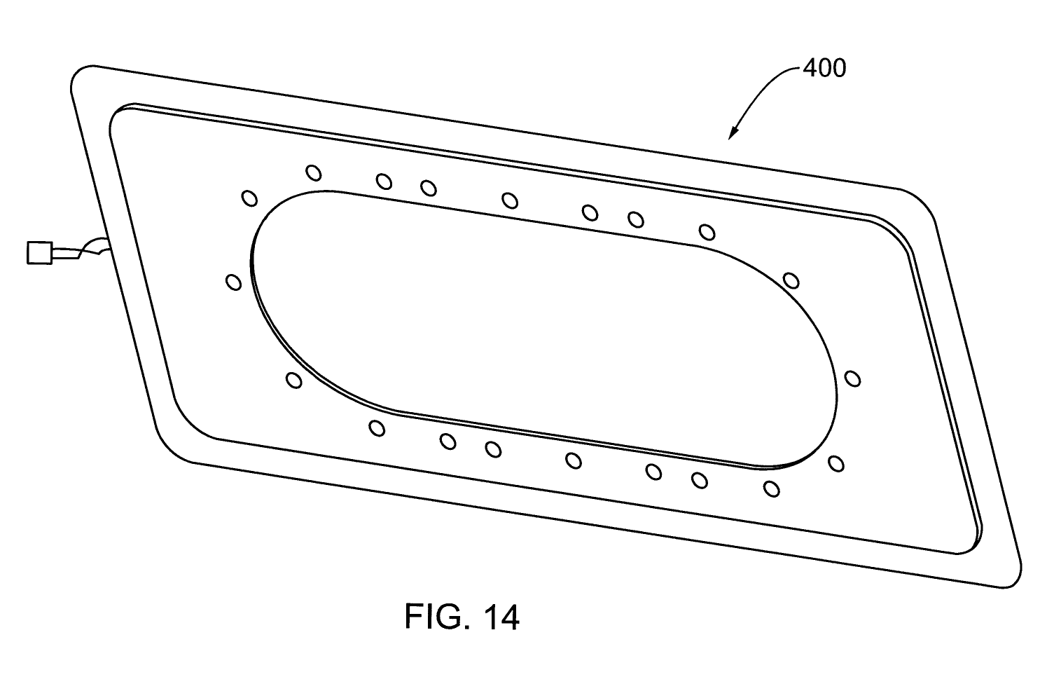

FIG. 14 shows one side of yet another phototherapy bandage 400. The phototherapy bandage 400 is very similar to phototherapy bandage 300. In this embodiment, the cut-outs formed in the adhesive layer and printed circuit boards are ovoid rather than circular making the phototherapy bandage 400 better suited for covering elongate wounds. Although FIGS. 13 and 14 show circular and ovoid cut outs, those of skill in the art will appreciate that cutouts having other geometric shapes (oval, crescent, square etc.) can be provided in the adhesive layer and printed circuit board.

Although the controller 54 is shown as comprising a wireless communications transceiver 142, if desired the controller may alternatively comprise a wireless communication receiver such as for example, an infrared receiver. In this case, the controller 54 is able to receive phototherapy treatment protocol programs from a remote device such as for example a personal digital assistant (PDA) or cellular telephone having an IrDA compatible infrared communications interface but is unable to transmit temperature, light and impedance data recorded by the temperature sensors, photoreceptors and contact sensors.

Although the phototherapy bandages are described and shown as comprising radiation emitting sources in the form of red LEDs 106, 324, those of skill in the art will appreciate that alternative radiation emitting sources may be employed. For example, radiation emitting sources that emit light at other visible wavelengths or at non-visible wavelengths, such as for example ultraviolet and near infrared wavelengths may be employed. The type of radiation emitting sources that are employed is selected for their therapeutic and/or energy properties. Longer wavelengths in the near infrared can have significant depth of penetration.

Ultraviolet radiation sources may be employed in order to stimulate a light emission response in nanocrystals. Nanocrystals (also called quantum dots) give off very narrow band light which is related to the physical size of the crystal. Wavelengths from violet to the near-infrared are possible by selecting the appropriate crystal size and positioning them near the ultraviolet radiation sources. Combining different sized crystals in a matrix can also provide unique spectral bandwidths of multiple wavelengths all emitting simultaneously. Alternately, the radiation emitting sources may comprise a matrix of nanocrystals which are aligned across a larger surface and sandwiched between two conducting media such that the flow of electrical current causes electroluminescence of the matrix.

In the embodiments described above, the phototherapy bandage comprises temperature sensors, photoreceptors and contact sensors. As will be appreciated by those of skill in the art, the phototherapy bandage need not include each of these sensors. Rather the phototherapy bandage may comprise a subset of the sensors or other sensors in addition to the temperature sensors, photoreceptors and contact sensors. Alternatively, the phototherapy bandage may comprise different sensors to sense other parameters indicative of wound healing.

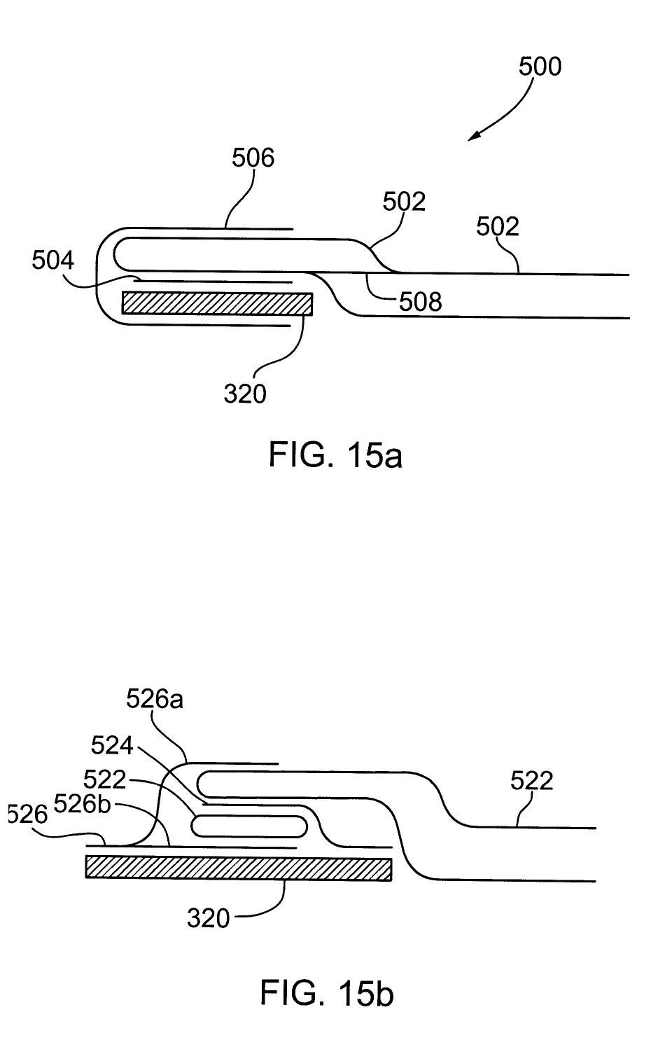

For example, turning now to FIG. 15a, a pressure sensor suitable for use with the phototherapy bandages 300 and 400 described above is shown and is generally identified by reference numeral 500. As can be seen, the pressure sensor 500 is partially embedded in foam dressing material 502 positioned in the cut-outs 322 and 332 and overlying the wound and comprises a sense electrode 504 surface mounted on one side of a portion of the printed circuit board 320 that has been extended into the cut-out region. The sense electrode 504 is separated from a reference electrode 506 by a portion of the dressing material 502. The dressing material 502 interposed between the sense and reference electrodes 504 and 506 respectively acts as an elastic dielectric. As a result, the sense and reference electrodes 504 and 506 respectively, form the plates of a parallel-plate capacitor. The reference electrode 506 is folded around the sense electrode 504 to shield the sense electrode from external noise and is surface mounted on the opposite side of the extended portion of the printed circuit board 320.

In this embodiment, the reference electrode 506 is formed of flexible conductive tape, ribbon, foil etc. that can be easily folded. A membrane 508 isolates the portion of the dressing material in contact with the wound from the portion of the dressing material separating the sense and reference electrodes. The dressing material 502 separating the sense and reference electrodes has a thickness in the range of from about ⅛″ to about ¼″.

As will be appreciated, when the dressing material 502 is subjected to pressure and compresses, the spacing between the sense electrode 504 and the reference electrode 506 changes resulting in a change in capacitance of the capacitor occurring. This change in capacitance is read by the controller 54 allowing the pressure applied to the dressing material 502 and hence, to the wound area to be determined.

Depending on the size of the wound and hence the size of the dressing material 502 applied on the wound bed, the number of pressure sensors 500 incorporated into the dressing material may vary.

FIG. 15b shows an alternative pressure sensor 520. In this embodiment, one end of the sense electrode 524 is trapped between two layers of foam dressing material 522. The other end of the sense electrode 524 undergoes a curve and is surface mounted on the top surface of the extended portion of the printed circuit board 320. The reference electrode 526 is also surface mounted on the top surface of the extended portion of the printed circuit board 320 and has a first arm 526a overlying the top layer of the foam dressing material 522 and a second arm 526b extending beneath the lower layer of the foam dressing material 522 to yield a layered capacitor configuration. Similar to the previous embodiment, the reference electrode 526 shields the sense electrode 524 from external noise. As will be appreciated, the layered capacitor configuration of pressure sensor 520 has improved sensitivity as compared to that of pressure sensor 500 but requires greater printed circuit board area.

Although the pressure sensors 500 and 502 have been described for use with the phototherapy bandages 300 and 400, those of skill in the art with appreciate that the pressure sensors may be used with the phototherapy bandage 52. In this case, access for the sense and reference electrodes to the printed circuit boards of the segments needs to be provided through the encapsulating material 112. Of course, the pressure sensors may be used in other bandage configurations where it is desired to measure and/or monitor the pressure being applied to a wound region.

Although embodiments have been described with reference to the drawings, those of skill in the art will appreciate that variations and modifications may be made without departing from the spirit and scope thereof as defined by the appended claims.

1. A phototherapy device comprising:
a plurality of radiation emitting sources arranged at spaced locations along at least a portion of the periphery of a wound to be treated; and
a controller communicating with and controlling operation of said radiation emitting sources.

2. A phototherapy device according to claim 1 wherein said controller illuminates said radiation emitting sources at intervals.

3. A phototherapy device according to claim 2 wherein said controller controls the intensity level of said radiation emitting sources during illumination.

4. A phototherapy device according to claim 1 said radiation emitting sources are light emitting diodes.

5. A phototherapy device according to claim 1 wherein at least some of said radiation emitting sources emit radiation having a wavelength in the range of from about 630 nm to about 690 nm.

6. A phototherapy device according to claim 1 wherein said radiation emitting sources are arranged at spaced locations generally about the entire periphery of said wound.

7. A phototherapy device according to claim 1 wherein said radiation emitting sources are embedded in a bandage sized to overlie said wound.

8. A phototherapy device according to claim 7 wherein said bandage comprises an upper breathable layer and a lower layer to contact a subject, the lower layer having a cut-out therein sized to accommodate said wound, said radiation emitting sources being trapped between said upper and lower layers.

9. A phototherapy device according to claim 8 wherein said lower layer has an adhesive thereon to affix said bandage to said subject.

10. A phototherapy device according to claim 8 wherein said radiation emitting sources are mounted on at least one printed circuit board trapped between said upper and lower layers.

11. A phototherapy device according to claim 10 wherein said radiation emitting sources are mounted on a single flexible printed circuit board and are positioned generally about the periphery of a cut-out formed in said printed circuit board that is sized to accommodate said wound.

12. A phototherapy device according to claim 10 wherein said radiation emitting sources are arranged in groups, each group of radiation emitting sources being mounted on an individual printed circuit board segment.

13. A phototherapy device according to claim 12 wherein adjacent segments are interconnected by a flexible conductive cable.

14. A phototherapy device according to claim 1 wherein said radiation emitting sources are spaced from the periphery of said wound by a distance in the range of from about 1 cm to about 3 cm.

15. A phototherapy device according to claim 1 further comprising at least one sensor proximate said radiation emitting sources and communicating with said controller.

16. A phototherapy device according to claim 15 wherein said controller reads the at least one sensor at intervals.

17. A phototherapy device according to claim 16 wherein said controller transmits read sensor values to one or more remote computing stations.

18. A phototherapy device according to claim 15 wherein said at least one sensor is selected from the group comprising a temperature sensor, a photoreceptor, an impedance detector and a pressure sensor.

19. A phototherapy device according to claim 18 wherein said at least one sensor comprises two or more sensors selected from the group comprising a temperature sensor, a photoreceptor, an impedance detector and a pressure sensor.

20. A phototherapy device according to claim 11 further comprising a plurality of temperature sensors positioned on said printed circuit board at spaced locations generally about the periphery of said cut-out.

21. A phototherapy device according to claim 11 further comprising a plurality of photoreceptors positioned on said printed circuit board at spaced locations generally about the periphery of said cut-out.

22. A phototherapy device according to claim 11 further comprising at least one impedance detector comprising a pair of contact sensors, said contact sensors being positioned on said printed circuit board at diametric locations relative to said cut-out.

23. A phototherapy device according to claim 17 wherein said controller comprises a wireless transmitter to transmit read sensor values over a wireless communications link.

24. A phototherapy device according to claim 16 wherein said controller comprises a processor executing at least one phototherapeutic regiment program that determines the illumination sequence of said radiation emitting sources and the reading sequence of said at least one sensor.

25. A phototherapy device according to claim 24 wherein said controller comprises a wireless receiver to receive one or more phototherapeutic regiment programs over a wireless communications link.

26. A phototherapeutic device according to claim 16 wherein said radiation emitting sources and controller are releasably connected via a physical link.

27. A phototherapy device according to claim 13 further comprising at least one temperature sensor positioned on each printed circuit board segment.

28. A phototherapy device according to claim 13 further comprising at least one photoreceptor positioned on each printed circuit board segment.

29. A phototherapy device according to claim 13 further comprising at least one a pair of contact sensors, said contact sensors being positioned on diametrically opposite printed circuit board segments.

30. A phototherapy device according to claim 8 further comprising at least one pressure sensor monitoring the pressure applied to said wound through said bandage.

31. A phototherapy device according to claim 30 wherein said at least one pressure sensor is a capacitive sensor.

32. A phototherapy device accordingly to claim 31 wherein said capacitive sensor comprises a sense electrode, a reference electrode and a compressible dielectric interposed between said sense electrode and reference electrode.

33. A phototherapy device according to claim 32 wherein said dielectric is a foam dressing material positioned in said cut-out and overlying said wound.

34. A phototherapeutic device according to claim 32 wherein said reference electrode shields said sense electrode from external noise.

35. A phototherapeutic device according to claim 30 wherein said controller reads the at least one pressure sensor at intervals.

36. A phototherapy system comprising:
at least one computing station; and
one or more phototherapy devices according to claim 1 communicating with said at least one computing station.

37. A phototherapy system according to claim 36 wherein said at least one computing station communicates with one or more of said phototherapy devices over a wireless communications link.

38. A method of treating a wound comprising irradiating the skin tissue adjacent the periphery of the wound with light energy at intervals.

39. The method of claim 38 further comprising monitoring the wound during said intervals.

40. A wound sensing device comprising:
a plurality of sensors for monitoring at least one wound parameter to be positioned adjacent a wound; and
a controller communicating with and reading said sensors.

41. A wound sensing device according to claim 40 wherein said sensors are selected from the group comprising temperature sensors, light sensors, impedance sensors and pressure sensors.

42. A wound sensing device according to claim 41 wherein said sensors are embedded in a bandage sized to overlie said wound.

43. A wound sensing device according to claim 42 wherein said controller reads said sensors at intervals.

44. A wound sensing device according to claim 43 wherein said controller transmits read sensor values to a remote computing location.

45. A wound sensing device according to claim 44 wherein said controller transmits read sensor values to said remote computing location over a wireless communications link.

46. A phototherapy bandage comprising:
an upper layer;
a lower layer; and
a plurality of spaced light emitting devices arranged in a ring and positioned between said upper and lower layers.

47. A phototherapy bandage according to claim 46 wherein said light emitting devices are arranged about the periphery of a cut-out formed in said lower layer, said cut-out being sized to accommodate a wound.

48. A phototherapy bandage according to claim 47 wherein said light emitting devices are light emitting diodes.

49. A phototherapy bandage according to claim 47 wherein said light emitting diodes are mounted on at least one printed circuit board trapped between said upper and lower layers.

50. A phototherapy bandage according to claim 49 wherein said upper layer is breathable.

51. A phototherapy bandage according to claim 50 wherein said lower layer has adhesive thereon.

52. A phototherapy bandage comprising:
an upper layer;
a lower layer; and
a plurality of spaced sensors arranged in a ring and positioned between said upper and lower layers.

53. A phototherapy bandage according to claim 52 wherein said sensors are arranged about the periphery of a cut-out formed in said lower layer, said cut-out being sized to accommodate a wound.

54. A phototherapy bandage according to claim 53 wherein said sensors are selected from the group comprising temperature sensors, light sensors, impedance sensors and pressure sensors.

55. A phototherapy bandage according to claim 53 wherein said sensors are mounted on at least one printed circuit board trapped between said upper and lower layers.

56. A phototherapy bandage according to claim 55 wherein said upper layer is breathable.

57. A phototherapy bandage according to claim 56 wherein said lower layer has adhesive thereon.
